(19) United States
(12) Patent Application Publication
(10) Pub. No.: US 2012/0288921 A1
(43) Pub. Date: Nov. 15, 2012

(54) SOLAR POWERED SPECTRAL PHOTOSYNTHETIC BIOREACTOR SYSTEM FOR CULTURING MICROALGAE AT HIGH DENSITY

(75) Inventors: Zhenhong Yuan, Guangdong (CN); Kang Yang, Guangdong (CN); Zhongming Wang, Guangdong (CN); Shunni Zhu, Guangdong (CN); Changhua Shang, Guangdong (CN); Weizheng Zhou, Guangdong (CN)

(73) Assignee: GUANGZHOU INSTITUTE OF ENERGY CONVERSION, CHINESE ACADEMY OF SCIENCES, Guangdong (CN)

(21) Appl. No.: 13/514,389

(22) PCT Filed: Jul. 27, 2010
(86) PCT No.: PCT/CN2010/075484

§ 371 Date: Jul. 19, 2012

(30) Foreign Application Priority Data
Dec. 10, 2009 (CN) 200910213692.7

(51) Int. Cl. C12M 1/42

(52) U.S. Cl. 435/287.1; 435/292.1

(57) ABSTRACT

A solar powered spectral photosynthetic bioreactor system for culturing microalgae at high density, the system comprising a photobioreactor and further comprising a solar collector, optical fiber, an illuminant device within the photobioreactor, and a residual gas absorption device and culture medium separation and recovery device each connected to the photobioreactor; the illuminant device having one end connected to a spectral light intensity adjusting device installed above the photobioreactor; the adjusting device connected to the solar collector via the optical fiber; a gas distributor provided between the underside of the illuminant device and a base part of the photobioreactor; the distributor connected to an output end of a gas mixing device. This system can effectively improve utilization of solar energy, lower external electrical power consumption, and resolve the problems of solar energy being intermittent and unstable in nature and difficult to collect, ensuring continuous and stable culturing of microalgae.

### FIELD OF THE INVENTION

The present invention relates to the field of microalgae culturing biotechnology, and specifically is a photosynthetic bioreactor system for culturing microalgae at high density, the bioreactor system working by using solar light and heat in a complementary manner.

### BACKGROUND ART

The ever-increasing pressure of the global resource shortage has been accompanied by the development and use of marine algae, which will be an important approach to a long-term resolution for sources of food and energy for people. Algae are not only rich in proteins, fats, and carbohydrates, which are the three major categories of substances needed by the human body, but also further contain various types of amino acids, vitamins, antibiotics, and high unsaturated fatty acids, as well as many other biologically active substances, and can be used as a source of important raw materials for food products, medicine, biochemical reagents, fine chemical products, fuel and other materials. As people's knowledge of microalgae deepens, research to develop and design novel high-efficiency photobioreactors and applications thereof with respect to high-density culturing of microalgae has become an important component of microalgae biotechnology.

Culturing microalgae primarily involves two types of photobioreactors—open and enclosed. Open photobioreactors are structurally simple, inexpensively made, and easy to operate, but suffer from such defects as being prone to contamination and lacking stable culturing conditions. Future development is now directed toward closed photobioreactors, which have stable culturing conditions, can be operated in a sterile manner, and readily allow for high-density culturing. Types of common closed photobioreactors include tubular, flat plate, columnar airlift, stirred-tank, and floating bag-type. At present, there are primarily two different modes of lighting in closed photobioreactors, these modes being direct outdoor lighting and artificial light source lighting. The use of outdoor lighting is susceptible to the impact of light, temperature, and other factors of the external environment, and is not advantageous in terms of controlling the microalgae culturing process; the use of an artificial light source makes it possible for the reactor to work indoors, thus avoiding the impact of environmental factors, but the high energy consumption of an artificial light source is not advantageous in terms of limiting the costs of large-scale culturing and production of microalgae.

Solar energy is the most abundant source of clean energy on Earth, and all sources of energy on Earth, inclusive of fossil fuels, are derived from the sun. At about 5.7×10²⁴ J each year, the amount of solar energy that strikes the surface of the Earth is about 10,000 times the amount of energy used by humans, and the amount of solar radiation energy intercepted by the Earth each year is equivalent to 1,500 times the current amount of global electrical energy. However, problems are presented such as in that solar energy is a low-density power source that is intermittent and unstable and is difficult to collect, and the large-scale utilization thereof has therefore been constrained. Moreover, in order for solar energy to be utilized, there needs to be an effective carrier, and the solar energy needs to be converted to an energy source that can be stored, transported, and continuously outputted.

### DISCLOSURE OF THE INVENTION

An objective of the present invention resides in providing a solar powered spectral photosynthetic bioreactor system for effectively improving the utilization of solar energy, the system being continuously and stably applied to culturing microalgae.

In order to achieve the foregoing objective, the present invention adopts the following technical scheme: a solar powered spectral photosynthetic bioreactor system for culturing microalgae at high density, the system comprising a photobioreactor and further comprising a solar collector, optical fiber, an illuminant device installed within the photobioreactor, and a residual gas absorption device and culture medium separation and recovery device each respectively connected to the photobioreactor; the illuminant device having one end connected to a spectral light intensity adjusting device installed above the photobioreactor; the spectral light intensity adjusting device being connected to the solar collector via the optical fiber; a gas distributor being provided between the underside of the illuminant device and the base of the photobioreactor; the gas distributor being connected to an output end of a gas mixing device; an input end of the gas mixing device being an access for a CO₂ gas source, an N₂ gas source, or a gas source of compressed air or the like, it being readily possible to accurately adjust the components of the gas source.

The gas distributor is disposed between an inner cylinder and the reactor, is linked to the gas mixing device, and uses an airlift loop mixing scheme to generate circular flow in the culture medium.

The illuminant device comprises a double-layer transparent inner cylinder installed coaxially with the photobioreactor, and a plurality of side-glowing optical fibers installed in the clearance between the double layers of the inner cylinder, a top end of the side-glowing optical fibers being connected to the spectral light intensity adjusting device. The illuminant device utilizes an optical fiber system having high light transmission efficiency, with side-glowing optical fibers used to illuminate the interior of the photobioreactor, the optical fibers being different from a typical optical fiber in that when a light source is inputted at a light-incidence end thereof, a side surface is able to emit a continuous and uniform light. A plurality of sections of the side-glowing optical fibers are vertically loaded between wall surfaces of the double layers of the inner cylinder along the axial direction of the inner cylinder and arranged in the photobioreactor together with the inner cylinder, following which it is possible to form uniform and effective light illumination conditions in the photobioreactor inside and outside the inner cylinder. This type of structure has a larger light illumination surface area to volume ratio and a shorter optical path by which light can be transferred to the microalgae. At the same time as when the top of the side-glowing optical fibers is connected to the optical fiber to allow in solar light, an LED light source is mounted onto the base of the side-glowing optical fibers; the side-glowing optical fibers can be used for illumination, thus avoiding a redundant layout of an auxiliary lighting system within the photobioreactor, this being advantageous in terms of simplifying the structure and ensuring an effective use of the volume of the photobioreactor. The two light sources employ the same illuminant device for illumination, and the solar light source and LED auxiliary light source are able to work independently or simultaneously.

Further comprised is an auxiliary lighting system, the auxiliary lighting system having one end connected to the solar collector and another end connected to the illuminant device. The system is provided with the auxiliary lighting system which converts solar energy to electrical energy for storage, and is able to effectively mitigate circumstances where the solar light changes and where there is a deficit of light intensity at night.

The auxiliary lighting system further comprises a successively connected solar panel, storage battery, LED auxiliary lighting device, and LED light source, the solar panel being installed above the solar thermal collector, the LED auxiliary lighting device being installed above the photobioreactor, and the LED light source being installed at the bottom of the illuminant device.

The solar thermal collector comprises a parabolic concentrator cooler, the parabolic concentrator cooler having an inner side on which a primary parabolic concentrator mirror is provided, a secondary concentrator mirror being installed on in the central axis of the solar thermal collector. An automatic solar tracker is provided on the secondary concentrator mirror, and the secondary concentrator mirror is connected to the optical fiber. Solar light undergoes spectral filtering and adjustment through a parabolic mirror, a filter, and a shutter, thus obtaining a spectral band matched to the microalgae absorption spectrum and increasing the effective light density. Reflective film characteristics of the primary parabolic concentrator mirror are such that visible light can be reflected and infrared light can be transmitted. Installing the parabolic concentrator cooler on the back surface thereof makes it possible to absorb the transmitted infrared light.

Further comprised is a heat exchange device, the heat exchange device having one end connected to the parabolic concentrator cooler and another end connected to the inside of the photobioreactor.

The heat exchange device comprises a heat accumulator, a circulation pump, and a heat exchanger, the parabolic concentrator cooler being connected to the heat accumulator, the heat exchanger being installed within the photobioreactor, and a heat exchanger circuit being constituted between the heat exchanger, the circulation pump, and the heat accumulator. A reflective film of the primary parabolic concentrator mirror of the solar collector has characteristics for reflecting visible light and transmitting infrared light. When solar light passes through the primary parabolic concentrator mirror, the reflective film reflects visible light and transmits infrared light. The concentrator cooler absorbs thermal energy of the transmitted infrared light, and the thermal energy is guided into the heat accumulator via a pipeline. The heat accumulator and the heat exchanger, which is disposed within the photobioreactor, use the circulation pump to forcibly circulate and exchange heat, thus controlling the temperature inside the photobioreactor and also fully making use of the energy in different spectral bands of sunlight.

Within the photobioreactor, there is further installed an automatic detection device, the automatic detection device being able to monitor in real-time, during the reaction process, the intensity of light, the pH, the dissolved oxygen content, and other process parameters, and to provide a user culturing the microalgae with full reference information. The automatic detection device can be connected to the heat exchanger, causing the heat exchange device to act in concert with the use of the automatic detection device and making it possible to achieve the effect of automatically controlling the reactor temperature, which is conducive in conducting the heat exchange process.

On the top end of the photobioreactor, there are provided a reactor feed port and exhaust port, and on the base end thereof there is provided a reactor discharge port; the residual gas absorption device is connected to the exhaust port, and the two ends of the culture medium separation and recovery device are respectively connected to the reactor feed port and the reactor discharge port. A pressure limiting valve is further installed on the exhaust port. Installing the pressure limiting valve makes it possible to ensure that the pressure inside the reactor will be sufficient and to discharge residual gas from the reaction in a timely manner.

The spectral light intensity adjusting device comprises an adjustable filter and an adjustable light intensity shutter.

Compared to the existing art, the present invention presents the following advantages: The device of the present invention uses devices for collecting, diffracting, and transmitting solar energy to utilize solar energy in the culture of microalgae. This system employs different approaches for light, power, and heat to be able to effectively increase utilization of solar energy and to reduce the consumption of external electrical energy. Substituting the artificial light source with a closed photobioreactor that draws solar light into the chamber reduces the electrical energy consumed by the closed photobioreactor and simultaneously makes use of a technologically mature form of photovoltaic cell—the LED light source auxiliary lighting—at times when solar lighting conditions are not sufficient, thus resolving the problems of solar energy being intermittent and unstable in nature and difficult to collect, ensuring the microalgae will be continuously and stably cultured.

### BRIEF DESCRIPTION OF THE DRAWINGS

FIG. 1 is an overall structural diagram of the system of the present invention;

FIG. 2 is a structure diagram of the solar collector of the present invention;

FIG. 3 is a structural diagram of the photobioreactor of the present invention;

FIG. 4 is a diagram of the connection between the LED auxiliary lighting device and the side-glowing optical fiber in the present invention; and

FIG. 5 is a structural diagram of the gas distributor of the present invention.

### DESCRIPTION OF THE REFERENCE NUMERALS

1—solar collector;
2—optical fiber;
3—photobioreactor;
31—inner cylinder;
4—heat accumulator;
5—circulation pump;
6—heat exchanger;
7—gas mixing device;
8—gas distributor;
9—culture medium separation and recovery device;
10—storage battery;
11—solar panel;
12—spectral light intensity adjusting device;
13—automatic detection device;
14—LED auxiliary lighting device;
15—primary parabolic concentrator mirror;
16—parabolic concentrator cooler;
17—automatic solar tracker;
18—secondary concentrator mirror;
19—side-glowing optical fiber;
20—inner cylinder support;
21—reactor discharge port;
22—reactor feed port;
23—exhaust port;
24—residual gas absorption device;
25—LED light source;
26—pressure limiting valve;
27—aerator.

### SPECIFIC EMBODIMENTS

The following incorporates the accompanying drawings and a specific embodiment to provide a more detailed description of the substance of the present invention.

### Embodiment

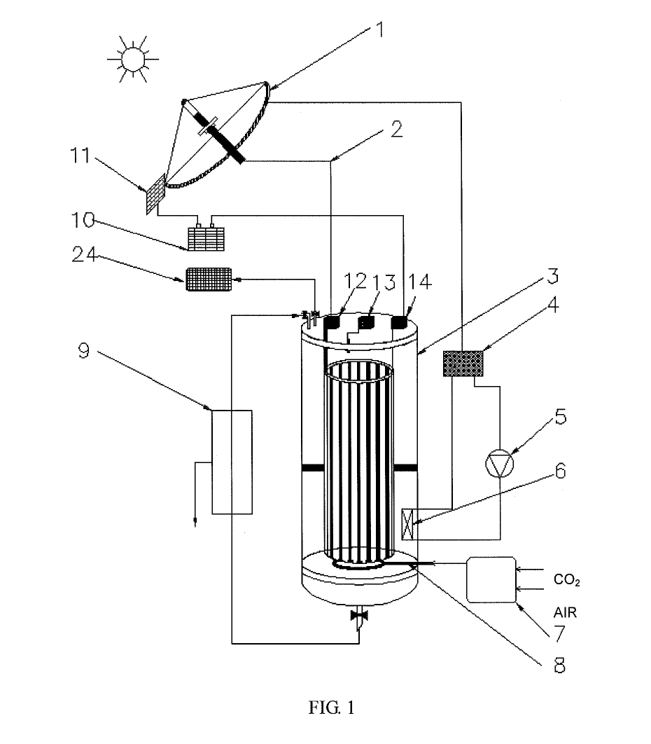

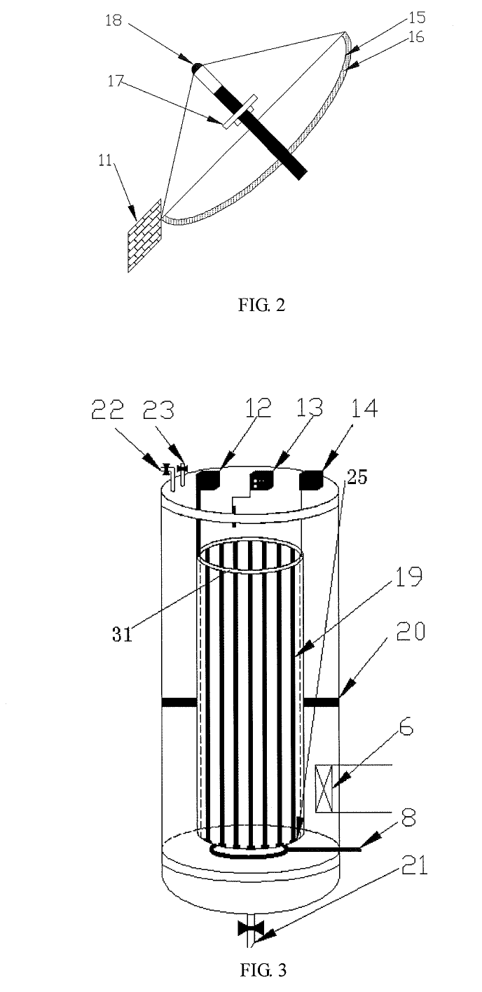

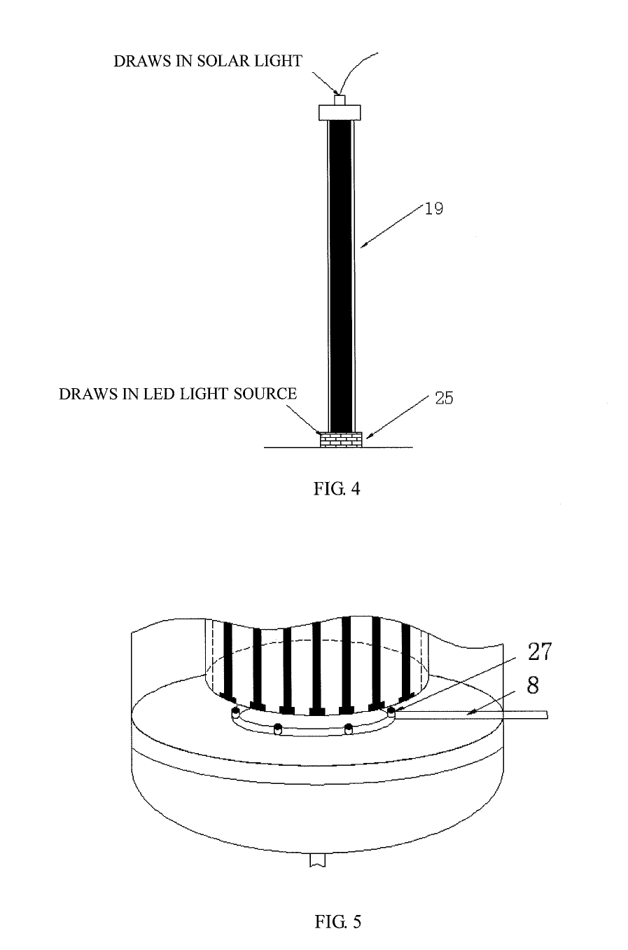

Reference is made to FIGS. 1 to 4, which illustrate a solar powered spectral photosynthetic bioreactor system for culturing microalgae at high density, the system comprising a photobioreactor 3 and further comprising a solar collector 1, optical fiber 2, an illuminant device installed within the photobioreactor 3, and a residual gas absorption device 24 and culture medium separation and recovery device 9 each respectively connected to the photobioreactor 3; the illuminant device having one end connected to a spectral light intensity adjusting device 12 installed above the photobioreactor 3; the spectral light intensity adjusting device 12 being connected to the solar collector 1 via the optical fiber 2; a gas distributor 8 being provided between the underside of the illuminant device and the base of the photobioreactor 3; and the gas distributor 8 being connected to a gas mixing device 7.

In the present embodiment, the photobioreactor 3 is made of a transparent PMMA (poly(methyl methacrylate), i.e., organic glass) cylindrical tank (the reactor being designed for a pressure of 0.2 MPa). An inner cylinder 31 formed of a double-layer transparent PMMA seal is further coaxially installed within the photobioreactor 3. The inner cylinder 31 is secured by an inner cylinder support 20 to the inside of the photobioreactor 3. A plurality of side-glowing optical fibers 19 are installed in the clearance between the double layers of the inner cylinder 31. The inner cylinder 31 and the side-glowing optical fibers 19 thus constitute an illuminant device. The top end of the side-glowing optical fibers 19 is connected to the spectral light intensity adjusting device 12, and the base end thereof is connected to an LED light source 25. The gas distributor is installed in the clearance between the base of the photobioreactor 3 and the base of the inner cylinder 31, and uses an airlift loop mixing scheme to generate a circular flow in the culture medium.

Further comprised is an auxiliary lighting system, the auxiliary lighting system having one end connected to the solar collector 1 and another end connected to the illuminant device. The auxiliary lighting system comprises a successively connected solar panel 11, storage battery 10, LED auxiliary lighting device 14, and LED light source 25. The solar panel 11 is installed onto the solar thermal collector 1 and is structurally integrated therewith. The LED auxiliary lighting device 14 is installed on the photobioreactor 3. The LED light source 25 is installed on the base end of the illuminant device.

The solar collector 1 comprises a parabolic concentrator cooler 16, a primary parabolic concentrator mirror 15 being provided on an inner side of the parabolic concentrator cooler 16 and a secondary concentrator mirror 18 being installed on the central axis of the solar thermal collector 1. An automatic solar tracker 17 is provided on the secondary concentrator mirror 18, and the secondary concentrator mirror 18 is connected to the optical fiber 2.

Further comprised is a heat exchange device, the heat exchange device having one end connected to the parabolic concentrator cooler 16 and another end connected to the inside of the photobioreactor. The heat exchange device comprises a heat accumulator 4, a circulation pump 5, and a heat exchanger 6. The parabolic concentrator cooler 16 is connected to the heat accumulator 4. The heat exchanger 6 is installed within the photobioreactor 3. A heat exchange circuit is thus constituted between the heat exchanger 6, the circulation pump 5, and the heat accumulator 4.

An automatic solar tracker 13 is further installed within the photobioreactor 3; this automatic solar tracker 13 can be connected to the heat exchanger 6.

The photobioreactor 3 is connected to the residual gas absorption device 24 and the culture medium separation and recovery device 9 via a reactor feed port 22 and an exhaust port 23 provided on the top end of the photobioreactor 3 and via a reactor discharge port 21 provided on the base end thereof. The residual gas absorption device 24 is connected to the exhaust port 23 and is used to absorb residual gas generated by the reaction in the photobioreactor 3. The culture medium separation and recovery device 9 has two ends respectively connected to the reactor feed port 22 and the reactor discharge port 21. A pressure limiting valve 26 is further installed on the exhaust port 23.

The present photobioreactor system is primarily used to culture microalgae and is specifically operated as follows.

Prior to the implementation of the microalgae culturing, saturated high-temperature steam is passed into the interior of the reactor through the feed port 22, and the high-temperature steam as well as high-pressure water are used to rinse and disinfect the reactor. A culture medium prepared in advance is pumped into the reactor through the reactor feed port 22, and microalgae strains are introduced therein. When lighting conditions are adequate, the solar collector 1 guides collected light through the optical fiber 2 into the spectral light intensity adjusting device 12; a beam achieves a spectrum adapted for microalgae growth after having passed through the spectral light intensity adjusting device 12, and enters into the photobioreactor 3, passing through the side-glowing optical fibers 19 placed sealed inside the clearance inside the inner cylinder 31 of the photobioreactor 3 to thus form uniform and effective lighting conditions within the photobioreactor 3. The solar panel 11 and the solar collector 1 adopt an integrated design and collect solar light while simultaneously the collected energy is stored in the storage battery 10; when lighting conditions are inadequate, lighting is provided by the LED auxiliary lighting device 14, which is linked to the storage battery, thus ensuring the lighting conditions within the reactor.

After the reaction has been allowed to proceed for a certain duration of time, culture medium rich in microalgae is released through the reactor discharge port 21 and passes through the culture medium separation and recovery device 9, thus obtaining microalgae, as well as the separated culture medium which is then recirculated into the photobioreactor 3 for repeated use.

Reference is made to FIG. 5, which illustrates a circular pipeline used by the gas distributor 8; above, a plurality of aerators 27 are uniformly distributed and linked to the pipeline, disposed in the clearance between the base of the photobioreactor 3 and the base of the illuminant device, thus making an inner loop as the mode of circulation. Such an airlift loop mixing scheme causes the culture medium to form a circular flow, thus acquiring favorable mixing of the materials and high intensity of gas-liquid mass transfer. At the same time, the shear force formed by the circulation is much lower than that of a forced circulation scheme using a circulation pump, and effects damaging the structure of the cultured microalgae are effectively reduced, which is appropriate for culturing microalgae having a lower tolerance for shear force.

The preceding detailed description is a specific illustration of a possible embodiment of the present invention, this embodiment in no way serving to limit the scope of protection of the present invention; the present scope of protection shall encompass any and all equivalent implementations or modifications not departing from the essence of the present invention.

1. A solar powered spectral photosynthetic bioreactor system for culturing microalgae at high density, the system comprising a photobioreactor, the system being characterized in further comprising a solar collector, optical fiber, and an illuminant device installed within the photobioreactor, as well as a residual gas absorption device and a culture medium separation and recovery device which are respectively connected to the photobioreactor;
the illuminant device having one end connected to a spectral light intensity adjusting device installed above the photobioreactor; the spectral light intensity adjusting device being connected to the solar collector via the optical fiber; a gas distributor being provided between the underside of the illuminant device and the base of the photobioreactor; and the gas distributor being connected to a gas mixing device.

2. The solar powered spectral photosynthetic bioreactor system for culturing microalgae at high density according to claim 1, characterized in that the illuminant device comprises a double-layer transparent inner cylinder installed coaxially with the photobioreactor as well as a plurality of side-glowing optical fibers installed in the clearance between the double layers of the inner cylinder, the top end of the side-glowing optical fibers being connected to the spectral light intensity adjusting device.

3. The solar powered spectral photosynthetic bioreactor system for culturing microalgae at high density according to claim 1, characterized in further comprising an auxiliary lighting system, the auxiliary lighting system having one end connected to the solar collector and having another end connected to the illuminant device.

4. The solar powered spectral photosynthetic bioreactor system for culturing microalgae at high density according to claim 3, characterized in that the auxiliary lighting system comprises a successively connected solar panel, storage battery, LED auxiliary lighting device, and LED light source, the solar panel being installed on the solar thermal collector, the LED auxiliary lighting device being installed on the photobioreactor, and the LED light source being installed on the base end of the illuminant device.

5. The solar powered spectral photosynthetic bioreactor system for culturing microalgae at high density according to claim 1, characterized in that the solar thermal collector comprises a parabolic concentrator cooler, a primary parabolic concentrator mirror being provided on an inner side of the parabolic concentrator cooler and a secondary concentrator mirror being installed on the central axis of the solar thermal collector, an automatic solar tracker being provided on the secondary concentrator mirror, and the secondary concentrator mirror being connected to the optical fiber.

6. The solar powered spectral photosynthetic bioreactor system for culturing microalgae at high density according to claim 5, characterized in further comprising a heat exchange device, the heat exchange device having one end connected to the parabolic concentrator cooler and having another end connected to the inside of the photobioreactor.

7. The solar powered spectral photosynthetic bioreactor system for culturing microalgae at high density according to claim 6, characterized in that the heat exchange device comprises a heat accumulator, a circulation pump, and a heat exchanger, the parabolic concentrator cooler being connected to the heat accumulator, the heat exchanger being installed within the photobioreactor, and a heat exchange circuit being constituted between the heat exchanger, the circulation pump, and the heat accumulator.

8. The solar powered spectral photosynthetic bioreactor system for culturing microalgae at high density according to claim 1, characterized in that an automatic detection device is further installed within the photobioreactor.

9. The solar powered spectral photosynthetic bioreactor system for culturing microalgae at high density according to claim 1, characterized in that a reactor feed port and exhaust port are provided on the top end of the photobioreactor, and a reactor discharge port is provided on the base end thereof, the residual gas absorption device being connected to the exhaust port, the two ends of the culture medium separation and recovery device being respectively connected to the reactor feed port and the reactor discharge port, and a pressure limiting valve being provided on the exhaust port.
